(19) United States
(12) Patent Application Publication
(10) Pub. No.: US 2005/0176090 A1
(43) Pub. Date: Aug. 11, 2005

(54) Method for flocculation of a fermentation broth comprising a fungus

(75) Inventors: Benjamin Raerup Knudsen, Saeby (DK); Prashant Iyer, Raleigh, NC (US)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 10/976,702

(22) Filed: Oct. 29, 2004

Related U.S. Application Data
(60) Provisional application No. 60/516,618, filed on Oct. 31, 2003.

(51) Int. Cl. C12N 9/62; C12P 1/00; C12N 1/16

(52) U.S. Cl. 435/41; 435/254.3

(57) ABSTRACT

A method for purifying an extracellular product of interest from a fungal fermentation broth comprising: a) subjecting the fermentation broth comprising a fungus to a fragmentation/disruption procedure; b) flocculating the fermentation broth; c) performing at least one separation step.

### CROSS-REFERENCE TO RELATED APPLICATIONS

This application claims priority or the benefit under 35 U.S.C. 119 of U.S. provisional application No. 60/516,618, filed Oct. 31, 2003, the contents of which are fully incorporated herein by reference.

### FIELD OF INVENTION

The present invention relates to a method for improving flocculation of a culture broth comprising a fungus. Additionally, the present invention relates to a method that improves downstream processing capacity.

### BACKGROUND OF THE INVENTION

In the industrial expression of a product of interest in fungi, optimization of the yield is a critical factor. This can be improved by improving the productivity of the production strain and/or improving the downstream recovery of the product of interest. Low recovery can be categorized as i) low flux during primary separation and secondary filtration, and ii) precipitates in filtrates and finished products. Primarily the problems are due to poor separation during drum filtration and the presence of fine particles in culture broth and inorganic precipitates which are carried on to down stream processing steps. Downstream processing steps could be improved by flocculation, which however is not well suited for fungi and especially filamentous fungi in which the biomass consists of a combination of highly structured parts, in the form of the mycelia, and to some degree of colloids which are difficult to remove by filtration. A need therefore exists for steps that can improve the downstream recovery of desired end products produced during fermentation in fungi.

### SUMMARY OF THE INVENTION

The present invention provides such an improvement of the downstream recovery by providing a fragmentation/disruption step before the flocculation step after harvest of the culture broth, so we claim:

A method for purifying an extracellular product of interest from a fungal fermentation broth comprising:
a) subjecting the fermentation broth comprising a fungus to a fragmentation/disruption procedure;
b) flocculating the fermentation broth;
c) performing at least one separation step.

### DETAILED DESCRIPTION OF THE INVENTION

The problems caused by poor separation during, e.g., drum filtration and presence of fine particles in the drum filtrate and inorganic precipitates which are carried on to down stream processing steps as described above, have in the present invention been addressed by introducing a fragmentation/disruption step before the flocculation step after harvest of a fungal fermentation culture. The flocculation step alone would have been difficult to carry out on fungal fermentation broths due to the large differences in particle size of the broth.

Conventionally attempts have been focused on improving the characteristics of the production strain thereby improving its flocculation properties.

In the present invention, however, good flocculation properties of the fungal cultures have been obtained by the introduction of a fragmentation/disruption step after the harvest of the culture broth from the fermentor.

In one embodiment the invention therefore relates to a method for improving flocculation of a fermentation broth comprising a fungus, wherein a fragmentation/disruption step is applied before flocculation.

The possibility of using flocculation on fungal fermentation broths will result in improvements on downstream processes and thus result in better yield of the end product and higher capacity during primary separation.

The present invention thus relates to a method for purifying an extracellular product of interest from a fungal fermentation broth comprising:
a) subjecting the fermentation broth comprising a fungus to a fragmentation/disruption procedure;
b) flocculating the resultant fermentation broth; and
c) performing at least one separation step.

The separation steps are conventional separation steps and comprise different types of filtrations and possible evaporation for further concentration.

Disruption/Fragmentation

Fungi and especially filamentous fungi in which the biomass consists of a combination of highly structured parts, in the form of the mycelia, and to some degree of colloids can according to the invention be fragmented into smaller pieces, small enough to be removed by filtration after flocculation.

The degree of fragmentation necessary depends on the robustness of the selected production strain. In one embodiment of the invention the right degree of fragmentation may be obtained by applying shear to the fermentation broth. Such shear would be sufficient to result in fragmentation, and in one embodiment the shear is provided by mixing, blending, and/or pumping.

Mixing and/or blending could be provided by any suitable mixer/blender the size of which may be adjusted according to the flow during operation. For small volumes like in lab-scale fermentations a handheld mixer or a kitchen blender may be used, and for bigger fermentation volumes in production scale mixers like, e.g., IKA Ultra TURRAX would be suitable. Also pumps are suitable for applying shear to fermentation broths and the capacity of the pump may as above be adjusted to the broth flow during operation. Examples of pumps suitable according to the invention are high shear pumps and dispersion pumps, such as IKA model UTL-150 for production scale and UTL-25 for pilot scale.

Other means for fragmentation according to the invention can be provided by, e.g., heating of the fermentation broth causing the fungal cell wall to disrupt and thereby leading to fragmentation. In one embodiment of the invention the fragmentation/disruption is therefore provided by heat. Heating should at least be above 34° C., and more particularly above 39° C. The upper limit would be determined by the desired end product and should be chosen so that the product will not be degraded or loose activity. It is to be noted that a heat treatment would normally require a holding time for getting the right degree of fragmentation.

In another embodiment the fragmentation/disruption is provided by enzymatic or chemical treatment. Examples of such enzymes or chemicals could be lysozyme.

The degree of fragmentation may be determined by examining a fragmented and a non-fragmented culture broth sample by, e.g., microscopy. The average length of the hypha of the fungus would typically according to the invention be reduced to less than 70% of the original length, in particular to less than 60% of the original length, preferably to less than 50% of the original length, more preferably to less than 40% of the original length, even more preferably to less than 30% of the original length.

Flocculation

After the fragmentation/disruption step the fermentation broth is preferably diluted 25-300%. The purpose of the dilution is to ease the flocculation by increasing the distance between suspended particles and thereby allowing the polymers (flocculation agents) to get in contact with the colloids. Too high dilution will, however, result in poor contact between colloids and polymer.

The flocculation agents are selected from the group consisting of salts and polymers. The flocculation agents may be cationic, anionic and/or non-ionic flocculation agents. Biomass, which normally is negatively charged, is first neutralized by the addition of, e.g., calcium chloride or aluminium salts. Calcium chloride works as a charge neutralizer due to its positive charge. When the biomass reaches a neutral net charge it will form agglomerates by hydrophobic interactions as a first step in the flocculation process. Calcium chloride should be added to a minimum of 0-5% v/v of a 36% solution. The effect of adding aluminium salts is similar to that of calcium, but aluminium salts also function as a colour binder. Aluminium salts also generate a lattice which works as an additional filter, though very weak. Aluminium salts may be added to 0-2% (v/v) of a 100% solution.

After biomass neutralization with calcium chloride or aluminium salts or both, the net charge is close to neutral. The cationic polymer binds these agglomerates into larger particles which then become positively charged. The cationic polymers may be added to 0-6% v/v of a 20% solution. The highly positive macro particles bound together with the cationic polymers are finally flocculated into larger macro particles with the anionic polymers, which may be added as needed, typically to 6-8% v/v of a 0.13% solution.

In one embodiment according to the invention the flocculating agent comprises GC850 (Gulbrandsen, S.C., USA), A130 (Cytec Industries, NJ, USA), C521 (Cytec Industries, NJ, USA), and CaCl₂.

In a further embodiment of the invention GC850 and C521 are combined. In a still further embodiment CaCl₂ and C521 are combined. In a still further embodiment CaCl₂ and C521 are combined and pH is adjusted to, e.g., pH 7.

When choosing a flocculating agent to be used according to the invention the pH of the fermentation broth has to be taken into account, since changes in pH will alter the net charge of both the biomass and the added chemicals. At lower pH the net charge becomes more positive, whereas at higher pH the net charge becomes more negative. The availability of chemicals for production therefore determines if pH changes are necessary.

In one embodiment the pH is not adjusted, and thus the pH is the broth pH. This is, e.g., the case when GC850 and heat curing are applied in the flocculation. In a further embodiment pH is adjusted. This is, e.g., the case when C521 and CaCl₂ are applied in the flocculation.

Thus in another embodiment the pH is adjusted to be comprised in the range of from pH 4 to pH 8, particularly from pH 5.5 to pH 7.0.

The method according to the invention is suitable for improving flocculation and for purifying products of interest, e.g., proteins produced in fungi. In a particular embodiment the fungi comprises filamentous fungi.

Fungus

The product of interest may be obtained from any fungus known in the art, particularly from any filamentous fungus known in the art.

In a preferred embodiment the product of interest may be obtained from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain, in particular the product of interest may be obtained from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderna viride strain.

In a particular preferred embodiment the product of interest may be obtained from Aspergillus, Humicola, or Trichoderma, preferably from an Aspergillus oryzae strain, from a Humicola insolens strain, or from a Trichoderma reseei strain.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

For purposes of the present invention, the term “obtained from” as used herein in connection with a given source shall mean that the valuable compound is produced by the source or by a cell in which a gene from the source has been inserted.

Product of Interest

The product of interest according to the invention is an extracellular product. It may be an antibiotic such as penicillin or cephalosporin or erythromycin, or a commodity chemical such as citric acid. The valuable compound may also be a polypeptide, in particular a therapeutic protein such as insulin, or an enzyme (e.g. a hydrolase, a transferase, a lyase, an isomerase, or a ligase, in particular a carbohydrolase, a cellulase, an oxidoreductase, a protease, an amylase, a lipase, or a carbohydrase).

The invention is further illustrated in the following examples which are not intended to be in any way limiting to the scope of the invention as claimed.

### EXAMPLES

### Example 1

Protocol and Process Conditions for the Flocculation Method.

The flocculation method according to the invention was tested on the production of amyloglucosidase produced in a filamentous fungus A. niger in a fed batch culture. After harvest drum filter flux with flocculation was compared to drum filter flux without flocculation and/or heat curing as pre-treatment.

During pre-treatment the following parameters were used:

The dilution (in tab water) was 150%, the addition of GC850 was added to 0.5% (v/v) of a 20% solution, the CaCl₂ concentration was added to 1.5% (v/v) of a 36 w/v % solution, and the A130 was added to 10% (v/v) of a 0.13 w/v % solution. NTU (nephelometric turbidity units) values ranging between 6-12.2 were measured during drum filter operation. In the part of the culture which was not subjected to flocculation before recovery the NTU ranged from 15-32, though NTU values from 30-60 have been observed for other batches. This shows that a reduction in turbidity results from the flocculation. The amyloglucosidase activity was also improved in the flocculated part of the culture by 16.5%.

Significant flux improvements during drum filtration were observed when flocculation was applied compared to fluxes without flocculation. This corresponds to an improvement between 38-44%.

A comparison of the product parameters showed no decrease in product quality.

From the trials performed with and without flocculation the general guidelines for the flocculation conditions can be worked out as given in Table 1 below:

TABLE 1

| Organism Fragmentation | Mechanical shearing/fragmentation with high |
|---|---|
| Harvest Curing/Chemical | shear dispersing pump/mycelia fragmentation |
| treatment | with heat curing or chemical. |
| Dilution H₂O (broth basis) | 25%-250% |
| Calcium salt (broth basis) | Minimum of 0-5% of a 36% sol. |
| Aluminum salt (broth basis) | 0-2% of a 100% sol. |
| pH adjustment | pH 4-8 |
| Cationic flocculation | 0-6% of a 20% sol. |
| chemical (broth basis) | |
| Anionic flocculation | As needed, typically 6-8% of a 0.13% sol. |
| chemical (broth basis) | |

Broth basis indicates that the percentages are calculated on the basis of the volume of the harvested culture broth.

### Example 2

Test of the Method According to the Invention in Large Scale.

The flocculation recipe was tested in large scale using the following batches B 1, B 2, B 3, and B 4, respectively. The high shear pump used was an ULTRA TURRAX type: ULT-150 NR: 95-1562 supplied by IKA—Machinenbau Janke & Knukel GmbH u. Co KG P79219 Staufen. Each of the cultures B1to B4 represent Aspergillus niger cultures expressing an extracellular amyloglucosidase and the cultures were fermented as fed batch cultures.

The following set-up was applied for all trials:

Process Diagram:

Harvest→Dispersion by high shear pump or dispersion pump→Pre-treatment→Drum filtration→Polish filtration→Germ filtration→Ultra filtration→Evaporation→Conservation and

Stabilization

In the above process diagram the dispersion by high shear pump or dispersion pump represents one embodiment of the fragmentation/disruption step according to the invention. Alternatively or as a supplementary treatment fragmentation can be obtained by heat curing or by enzymatic or chemical treatment. The pre-treatment step comprises flocculation.

CaCl₂ was not added to the culture broth during harvest, and recovery was started 5-10 hrs after harvest. Dilution during pre-treatment was 200% and the temperature was not adjusted, pH was adjusted to 7.1 by phosphoric acid or sodium hydroxide. CaCl₂ during pre-treatment was added to 2.1% (v/v) of a 36% solution and the C521 concentration was added to 1.8% (v/v) of an 18% solution. Drum filtration was performed on a 36 m² drum filter. Perlite decalite 4208 (Dicalite) was used as pre-coat on the drum filter and body feed, filter aid added during the process, was between 0-8%. Spray water was set to 2.0 m³ and drum filter rotations to 20 rpm. A130 dosing was 6.2% of a 0.13% (w/v) solution. The pH in the drum filtrate was adjusted to 4±0.2 with phosphoric acid or sodium hydroxide. Celite 512 (World Minerals) was used as pre-coat and body feed during polish filtration, the filtration temperature was kept at 35° C. HS 200 filter pads (Begerow) were used for germ filtration and the filtrate temperature was kept at 5° C. Concentration was performed by continuous ultra filtration to a refractive index of 25 (RI 25) with a concentrate temperature kept below 10° C., followed by evaporation to RI 47 with an evaporation temperature of 36° C.

The liquid product produced in these trials have not been ripened, hence no second time filtration has been employed in full scale. This was performed in pilot scale.

Up-Scaling Aspects

One of the key parameters found in lab trials was the level of fragmentation before pre-treatment. During lab trials a kitchen blender was applied to insure an even size distribution of the culture broth. During large scale trials an ULTRA TURRAX dynamic mixer was applied for cutting the branched structure of the fungi down to an even size distribution. In both cases the following pre-treatment and flocculation performed well. During large scale experiments a better mixing was achieved, because of the larger amount of shear stress from pumps, piping etc. and better filtration, e.g., drum filter for production scale and filter paper for lab scale. This was observed as lower NTU values for filtrates than those observed in lab trails.

The robustness of the flocculation is a critical aspect in the recovery process. The robustness of the flocculation seen during these trials appeared fine judged by the quality of the drum filtrate and the high fluxes achieved.

Filtration of flocculated broth was tested in lab-scale, where high fluxes were observed. Despite the difference in equipment setup from lab to production scale, high fluxes were also observed during drum filtration on all batches. NTU was monitored in the drum filtrate as an evaluation of the efficiency of the filtration compared to lab-experiments. This shows that the method is scalable.

The most noticeable difference observed during the drum filtration was the efficiency of the flocculation for removing fine particles and other biomass fragments during filtration. Drum filtration without flocculation shows that the coat is slowly penetrated by biomass fragments and other fine particles. This eventually gives poor quality of the filtrate and also decreases the degree in which the drum filters can be utilized.

In contrast when flocculation was applied during trials no penetration of the coat with biomass fragments was observed. Because the porous structure of the pre-coat was kept open and not fouled by biomass fragments, the impact on capacity was extensive. This high removal of biomass fragments and other fine particles insured low NTU in the filtrate thereby enabling easier filtrations downstream.

During all flocculation trials the capacity and broth flux were challenged on the drum filters. Drum filter fluxes without fragmentation and flocculation were compared to drum filter fluxes according to the invention. Trials were performed on a 36 m² drum filters and the fluxes were gradually increased. Through normal recovery (without fragmentation and flocculation) the average culture broth flux varies between 40-60 L/(m²h). In the present trials the maximum culture broth flux achieved on the drum filter was 125 L/(m²h) over a period of 1½ hours with steady levels in the drum vessel. In all trials it was possible to reach culture broth fluxes of 110 L/(m²h). This corresponds to an improvement in flux of at least 90%.

Although an improved capacity and a higher quality of filtrate were observed, overdosing of flocculent A130 may to some extent affect the ability of the flocculated broth to stick to the drum pre-coat. The appropriate amount has to be decided on a case by case basis.

Using the fluxes observed in the present trials the average process time (pre-treatment and drum filtration) was calculated to 14.90 h. Compared to the normal process (no flocculation) the pre-treatment and primary separation time are reduced by a minimum of 10 hrs using the average values, this corresponds to a reduction of 40%.

The subsequent polish filtration, on primus (blank filter) and germ filtration on HS 200 filter pads, performed well. Fluxes were generally kept high, between 10-20 m³/h. No problems were observed during polish or germ filtration for the trials, mainly because of the low NTU values in the drum filtrate. Lowering the pH in the drum filtrate (from pH 7.1 to pH 4.0) did not trigger any precipitation of fatty acids or inorganic salts. Further, no problems were encountered during ultra filtration or evaporation. A too large pH adjustment with diluted phosphoric acid during stabilization can trigger jelly-like precipitates presumably denatured protein. This was observed on batch B 3, though without any noticeable yield loss.

During all trials enzyme activities were measured per unit operation for calculation of step yields. The average accumulated yield was improved by the method according to the invention, corresponding to a yield improvement of around 10%.

The increase in yields observed could be explained by less physical loss in sludge from drum filters or as reduced activity in ultra filtration permeates. The sludge removed during drum filtration of flocculated broth was easier to drain, i.e. the porous structure of the flocculated broth appeared more open.

Total process savings are presented in Table 2

TABLE 2
Estimated reduction possible by introducing flocculation on
amyloglycosidase using the method of the invention.

| | Reduction (%) |
|---|---|
| Reduction of filter aid consumption | 58 |
| Reduction in water and waste water | 52 |
| consumption | |
| Increase in yields | 10 |
| Reduced quantity for ultra filtration | 33 |

1. A method for purifying an extracellular product of interest from a fungal fermentation broth comprising:
a) subjecting the fermentation broth comprising a fungus to a fragmentation/disruption procedure;
b) flocculating the fermentation broth;
c) performing at least one separation step.

2. The method according to claim 1, wherein the fragmentation is provided by applying shear to the fermentation broth.

3. The method according to claim 1, wherein the fragmentation/disruption is provided by heat.

4. The method according to claim 1, wherein the fragmentation/disruption is provided by an enzymatic or a chemical treatment.

5. The method according to claim 3, wherein the fermentation broth is heated to above 34° C.

6. The method according to claim 3, wherein the fermentation broth is heated to above 39° C.

7. The method according to claim 2, wherein the shear is provided by mixing, blending, and/or pumping.

8. The method according to claim 1, wherein the flocculation is provided by adding a flocculation agent.

9. The method according to claim 8, wherein the flocculation agent is selected from the group consisting of salts and polymers.

10. The method according to claim 1, wherein pH of the fermentation broth is adjusted to a pH in the range of from pH 5.5 to pH 7.0.

11. The method according to claim 1, wherein the fungus is a filamentous fungus.

12. The method according to claim 11, wherein the filamentous fungus is selected from the group consisting of Acremonium, Aspergillus, Fusarium, Humicola, Mucor, Myceliophthora, Neurospora, Penicillium, Thielavia, Tolypocladium, and Trichoderma.
